(19) United States
(12) Patent Application Publication
(10) Pub. No.: US 2011/0071448 A1
(43) Pub. Date: Mar. 24, 2011

(54) LIQUID IMPERMEABLE MATERIAL

(76) Inventor: Ezio Margiotta, New South Wales (AU)

(21) Appl. No.: 11/917,357

(22) PCT Filed: Jun. 24, 2005
(86) PCT No.: PCT/AU05/00929

§ 371 Date: Sep. 1, 2009

(30) Foreign Application Priority Data
Jun. 24, 2004 (AU) 2004903435

(51) Int. Cl. A61F 13/00; B32B 3/24; B32B 27/32; B32B 9/02; B32B 25/08; B32B 27/08; B32B 27/06; D03D 9/00; B28B 1/48; C08J 9/00; A43B 5/02; A43B 23/02; A41D 19/00; A63B 43/00; A61F 5/01

(52) U.S. Cl. 602/5; 428/131; 428/220; 428/473; 428/492; 428/515; 428/518; 428/500; 442/1; 264/154; 521/50; 521/82; 521/134; 521/139; 36/128; 36/47; 2/167; 473/569; 602/47

(57) ABSTRACT

A liquid impermeable material comprising a layer of vapour permeable viscoelastic polymer gel, applications of said material, a composite material comprising said material and a method of producing said material.

The present invention relates to a liquid impermeable material that is also vapour permeable. In particular, the present invention relates to various applications of said material on its use in the construction of various goods.

### BACKGROUND OF THE INVENTION

There are various different materials commercially available which provide liquid impermeability that are composed of various constituents such as plastics, nylon, leather and canvas. Furthermore, there are various composite materials available which have a liquid impermeable layer adhered to another layer composed of a material that provides greater comfort when worn adjacent the skin.

However, in many clothing and apparel applications, such as for example sporting footwear, there is a need for a liquid impermeable material that is also vapour permeable. Furthermore, there is a need for a material that also provides the added characteristic of being able to absorb loads and dissipate shear forces.

It is envisaged that such a material could be used for a variety of applications where a material has the requirement of liquid impermeability in addition to vapour permeability and is subject to various load and shear forces, such as for example on car seat covers and swim wear applications.

Another technical area where a liquid impermeable but vapour permeable material would be desired in the medical industry. A material which can be present adjacent the skin, has hypoallergenic properties, and which is liquid and vapour permeable would be very beneficial for lining casts and bandages. Here the material would allow the skin of the patient to breath, whilst still providing a liquid impermeable layer.

Present materials that are currently used in the medical area which provide vapour permeability and liquid impermeability such as GOR-TEX have various disadvantages. In particular, GOR-TEX is stiff and non-conforming when used as a bandage or lining for a plaster cast. Furthermore, GOR-TEX has a poor elastic memory and as a result bottoms out fairly easily without returning to its original shape.

Surprisingly, it was found that by using various methods to produce a viscoelastic polymer gel sheet, a liquid impermeable material was produced that was also vapour permeable. The resultant material reduces the impact of loads and shear forces passing through the material with substantially little permanent deformation. Furthermore, it was surprisingly found that if this viscoelastic polymer gel was bonded to another material, a composite material could be formed which could be applied to many different applications that required a liquid impermeable material that was also vapour permeable.

### SUMMARY OF THE INVENTION

According to one aspect the present invention provides a liquid impermeable material comprising of a layer of vapour permeable viscoelastic polymer gel.

According to one aspect of the invention, the vapour permeability of the material may be provided by mechanically perforating the viscoelastic polymer gel layer. Preferably, the mechanical perforation consists of microperforations of 50 to 400 microperforations per cm² in the viscoelastic polymer gel and more preferably 150 to 300 microperforations per cm². Preferably, the diameter of the microperforations is in the order of 10 to 150 μm and more preferably 50 to 100 μm.

Alternatively, or in combination, a foaming agent or nucleating agent may be incorporated into the viscoelastic polymer gel during formation. The foaming agent or nucleating agent has the effect of producing discreet voids within the viscoelastic polymer gel layer providing passages for vapour to pass through whilst maintaining liquid impermeability. Preferably, the viscoelastic gel includes 0.5-5 wt % of foaming or nucleating agent.

In another alternative, or in combination with the above, a molecular sieve may be incorporated into the viscoelastic polymer gel during formation. Through the incorporation of the molecular sieve, the viscoelastic polymer gel inhibits liquid from passing through the second layer whilst maintaining vapour permeability. With the augmentation of a molecular sieve, a three dimensional interconnecting network is introduced to the gel material which preferentially or selectively pulls in and through water vapour molecules.

Preferably, the thickness of the viscoelastic polymer gel layer is in the order of 0.25 mm to 10 mm thick and more preferably 0.5 mm to 3 mm thick.

The composition of the viscoelastic polymer gel included in the material of the present invention preferably includes:
(a) 45% to 100% wt major polymer component A, and
(b) optionally 5% to 45% wt minor polymer component B.
wherein major polymer component A is chosen from any vinyl or polyolefin polymer, or combination thereof, that forms a viscloelastic polymer gel, and component B is chosen from any diene polymer or polyvinyl alcohol.

Preferably, major polymer component A is chosen from any linear or branched polyolefin group, such as for example ethylene, styrene or propylene, or a combination thereof. More preferably, the major polymer component A consists of up to 80% ethylene propylene styrene block copolymers and mineral or carrier oils ionically polymerised.

Preferably, minor polymer component B is chosen from polyisoprene.

Preferably, the viscoelastic polymer gel has a Shore Hardness value in the range of 25 to 75. Preferably the tensile strength of the viscoelastic polymer gel is greater than 60 psi. Preferably, the ultimate elongation of the viscoelastic polymer gel is between 250 and 1700% and preferably the tear strength is greater than 8 pounds per inch.

Preferably, the material of the present invention may be used as a lining in medical and/or therapeutic applications requiring a material that is skin conditioning and/or hypoallergenic, such as for example linings for the skin underneath a cast, bandages, adhesive bandages and the like.

According to another aspect of the material of the present invention may be used in the construction of gloves, and in particular disposable gloves.

According to further aspect the material of the present invention may be used in the construction of swimwear.

According to another aspect the present invention provides a liquid impermeable composite material which includes:
(a) a first layer; and,
(b) a second layer consisting of the vapour permeable viscoelastic polymer gel material.

Preferably the viscoelastic polymer gel material is bonded to the first layer.

The first layer may be composed of any natural or man made material, which may be used in the textile industries, such as for example, plastics, leather, vinyl, cotton, synthetic microfibres, rubber and wool. Preferably, the first layer is composed of a material that is also vapour permeable.

Preferably, the first layer is composed of leather, and more preferably, the first layer is a premium leather such as for example kangaroo leather.

The composite material can behave life a modified monolithic film membrane that allows transmission of vapours through facilitated diffusion. The permeant or liquid can dissolve/evaporate at the surface of the viscoelastic polymer gel on the side of the highest temperature (namely the side where the skin is, and hence via thermodynamics be driven or moved outwards away from the skin). Then any remaining vapour will diffuse across the membrane and out via the first layer.

According to a further aspect, the material of the present invention may be bonded to various films or scrims of a hydrophobic nature, such as polyester, polyurethane and polyethylene.

According to another aspect, the present invention provides an article of footwear including an upper portion composed of a liquid impermeable material that includes
(a) a first layer; and
(b) a second layer consisting of a vapour permeable viscoelastic polymer gel.

Preferably the viscoelastic polymer gel is bonded to the first layer. Preferably, the article of footwear is specifically designed as a football boot for use in relation to soccer, rugby union, Australian rules football, rugby league, American football and the like, or any type of football which involves a large amount of impact loads and shear stresses being applied to the foot surrounded by the upper portion of the article of footwear. Preferably, the first layer is composed of a premium leather material such as Kangaroo leather.

According to a further aspect, the present invention provides a ball for use in playing sport including an outer surface portion composed of a liquid impermeable material that includes
(a) a first layer; and
(b) a second layer consisting of a vapour permeable viscoelastic polymer gel.

Preferably the viscoelastic polymer gel is bonded to the first layer.

The ball maybe chosen from any type of ball which is used in a sporting activity. Preferably, the ball is one which is used in a sport wherein the ball is subject to high shear stresses, loads and sometimes wet environments, such as for example in soccer, rugby union, Australian rules football, rugby league, American football and the like.

According to another aspect, the present invention provides a method of producing a liquid impermeable material characterised whereby, the major polymer component A (viscoelastic gel) is heated to assume the molten state generally between 110° C. to 250° C. and preferably at between 160° C. to 210° C. for some 5 to 10 minutes.

Anhydrous lanolin can be added as a plasticiser to reduce viscosities further and distribute it thoroughly through the polymer melt at between 1% to 5% by weight.

Minor Polymer component B is added (pre liquefied) and may be chosen from such polymers as polyisoprene for greater strength or compression resistance (or other diene polymers). Or alternatively, polyvinylalcohol in gel solution of between 5% to 35% for more or less separation and channelling formation within the gel material, which seems to facilitate molecular sieve entrainment through the composition.

Major polymer component A may consist of up to 80% ethylene propylene styrene block copolymers and mineral or carrier oils ionically polymerised. These polymers are mixed with the mineral oils or resins until a viscous liquid is formed, and the polymers then swell to then form a viscoelastic polymer gel. Once the mixture returns to room temperature forms a gel.

Preferably, the blend is completely mixed in the liquefied state for a further 10 to 20 minutes at between 160 C to 200 C. Polymer A to B ratio is approximately 80:20. If desired, a 3A molecular sieve may be added towards the end of this blending phase in the proportion of approximately 10% to 20%.

At this point a nucleating agent may be added such as Hydrocerol CF in the amount of 0.5% to 20% by weight, and the blending continues further until maximum gas yield is reached. Usually, 1 to 5 minutes later.

The blend may then be poured onto a tray/platform and spread/rolled out to the desired thickness where it will set to room temperature after approximately 20 minutes later. The material may then be treated as a membrane and applied to any of the various uses within the ambit of the present invention.

It should be noted also that production processes/expertise could also apply the hot material to the preferred mould/application directly.

Alternatively, when set in the final stage, a specifically designed roller or press with fine gauge 0.3 mm or less spines whose depth of penetration is predetermined passes over the material in such a way as to create micropores.

According to another aspect, the production phase involves a second step heating of the blended polymer material. Here the liquid impermeable material is allowed to proceed and set as described above, then reheated to between 140° C. to 200° C. again for a second gas yield/nucleating affect to occur.

This process provides in increased density of cells/pores being created with smaller voids. Along with this embodiment the Molecular sieve can be additionally layered/spread or dusted over the material before this final second phases heating to set the molecular sieve to the outside and inside of the material.

### BRIEF DESCRIPTION OF THE FIGURES

The present invention will become better understood from the following detailed description of a preferred but non-limiting embodiment thereof, described in connection with the accompanying drawings, where in:

FIG. 1 is a schematic diagram showing a section of a liquid impermeable composite material in accordance with one aspect of the present invention;

FIG. 2 is a schematic diagram depicting a cross section of the surface of a sports ball;

FIG. 3 is a schematic diagram of an article of footwear in the form of a football boot;

FIG. 4 is a schematic diagram of an article of footwear in the form of a football boot;

FIG. 5 is a schematic diagram of a device for providing perforations into viscoelastic polymer gel layer, and

FIG. 6 is a schematic diagram of a textured viscoelastic polymer gel material.

### DETAILED DESCRIPTION OF THE INVENTION

The viscoelastic polymer gel is preferably composed primarily of an oil based polymer gel and preferably includes at least 5% w/w anhydrous lanolin. Examples of suitable oil based polymers include: soft touch viscoelastomer number 4125 produced by Gel Concepts of Whippany N.J. USA or Crinnis Corporations Soft and Medium Elastomer formulations; polydimethylsiloxanes and thermoreversible polymers produced by Kion Corporation NY USA; and polymethylmethacrylate gels produced by suppliers such as Sigma Aldrich USA.

Vinyl and polyolefins polymers in the viscoelastic family, such as ethylene, styrene and propylene can be blended and copolymerised and when formulated with carrier oils that are hypoallergenic and inert are ideal for the said material. Hydrophilic hydrophobic block copolymers may also be utilised within the scope of the present invention to further facilitate movement of water vapour whilst maintaining excellent bonding and mechanical properties of the viscoelastic polymer gel.

Depending on the desired softness, elongation capacity, melt temperature, tear strength, weight and specific gravity required, these components will vary from equal amounts to one or two polymers major proportions to one polymer minor proportion.

Anhydrous lanolin up to 5% or other natural oils such as jojoba, almond and various nut oils in 5% with 1-5% essential oils for odour reduction may be included in the mix. All such components are ideal being non-toxic and non-hazardous. They are inert and hypoallergenic unlike many rubbers, latex and silicones.

The supply of copolymers in basic form can be derived from suppliers as Crinnis Corp. USA or Gel Concepts NY USA.

The vapour permeability of the viscoelastic polymer gel may be provided by various methods either on their own or in combination. One such method is to mechanically perforate the viscoelastic polymer gel once it has been formed into a sheet of desired thickness. The perforations are from 10 to 150 μm in diameter and they may be provided at 50 to 400 perforations per square cm.

Another method is to provide a nucleating or foaming agent into the viscoelastic polymer gel during formation. A further method is to include a molecular sieve into the viscoelastic polymer gel during formation. Molecular sieves are crystalline metal alumina-silicates. When incorporated into the viscoelastic gel, they result in tetrahedra three-dimensional network, with a high internal surface area in which various gases and liquids are adsorbed. For present purposes a 3A molecular sieve is preferable as this selectively targets water vapour molecules.

Various additives may be included during the formation of the viscoelastic polymer gel for differing specific applications.

Additives could include colourants, dyes and pigments for altering colours and effects. Fire retardants with a decomposition temperature below the polymer material will reduce and control flame speed, size and heat, as well as smoke opacity and toxicity in the event of a fire.

Such appropriate fire retardants may include non halogenated additives, melamine derivatives, intumescents and inorganic retardants. This would permit use of the material in such applications as aircraft and automotive seat upholstery.

Microencapsulated Phase Change Materials (mPCMs) when introduced up to 50% by weight into the polymer blend adds to the utilisation of the material by the storing of thermal energy when changing the phase of the entrained material in relation to the prevailing ambient temperature. Useful temperature ranges may include but not be limited to 4-8° C., 18-25° C. and higher. When blended into the viscoelastic gel at formation the mPCM's permit superior heat capacity and heat transfer increasing or maximising comfort and safety minimising potential damages in the various applications the material may be used for.

In addition, anti-microbial agents may be included into the material, particularly when the material is used in medical and therapeutic applications.

Sheets would typically be produced by knife over air or roller mechanisms or roller coating apparatus. But may also be produced by casting sheets and extrusion moulding. The thickness of the sheets produced may be between 0.25 mm and 10 mm thick, but preferably, the thickness is in the order of 0.5 mm and 3 mm and this thickness provides the optimum vapour permeability to the layer of viscoelastic polymer gel.

A lesser capital expensive and highly flexible system was sought to produce the material in commercially viable sizes and quantities. The method developed involves incorporating a hot melt tank and blender/mixer with heated lines, a pumping mechanism and a variable width die with a doctor knife attached behind it and/or a traversing mechanism over a casting bed, with a cooling bed and a stacking station.

Sheet sizes, thickness and batch runs would only be limited by the capacity of the hot melt tank, pump speed die head size and casting bed size. After the components are melted, mixed and thoroughly heat blended in the tank and casting temperature is reached typically at around 210° C., a servo controlled hot melt casting die delivers the material to the casting bed where it is spread out to the desired thickness. The cooling sheet stations are modular and on wheels to facilitate relocation without interruption to the casting operations.

The machine can also be utilised to coat the material in molten state onto any flexible flat substrate, such as fabric, film, metal foil, leather, release papers/films/laminates and can be held onto the casting bed via vacuum. These films, substrates and papers can be utilised to create a textured surface on the material to assist feel, hydrodynamics and comfort.

When an extra dry surface is required the material can be dusted with an inert powder or talc that is hypoallergenic over the cooling station.

Once set the material can be microperforated at a size and density to facilitate vapour permeability and breathability. Such microperforation techniques are achieved with rollers over the material as supplied by such technologies as Burckhardt Basel Switzerland.

It is proposed that in a further embodiment the present invention provides a swimsuit or bodysuit be constructed by either bonding/heat seaming or welding panels of the material from flat sheet form or in the more elaborate method of blow moulding/casting or rotational moulding. Such patterns/forms may be obtained from either conventional custom fitting for skin tight clothing or Digital 3D body scanners.

The material may be produced with a microtextured surface at water contact, to assist or optimise hydrodynamics whilst the naturally compressive and elastic properties not only improve fit and comfort but also act as an active wall enhancing muscle contractions whilst maintaining the most natural streamlined shape the swimmer possesses.

Unlike other materials used today the material of the present invention may act like natural dolphin skin possessing emollients that act with water to further assist hydrodynamics. Further, unlike other fabrics used after 2 or 3 minutes they become soaked/wet whereas the material of the present invention cannot absorb water maintaining optimal predictable buoyancy capacities.

As the material may be formulated with fire retardants that meet industry standards, another embodiment would be the utilisation of the material for seat cushioning or lining, or for that matter any type of upholstered surface.

This is because the gel material can act as an ideal material to suppress noise and vibration along with being supremely conforming and hence increasing occupant comfort and safety. Particularly in the premium flat bed/seats in aircraft design and cabins and prestige automotive and marine markets where it accompanies both leather and woollen substrates for example, which are both highly sought and used.

In another embodiment of the present invention the material may be used as a lining in medical and/or therapeutic applications requiring a material that is skin conditioning and/or hypoallergenic, such as for example linings for the skin underneath a cast, bandages, adhesive bandages and the like. The material of the present invention has distinct advantages over other known materials such as GOR-TEX as the material of the present invention has good abrasion resistance, it is highly conforming for use in bandage applications, it has better elastic memory and absorbs shear stresses. Furthermore, by incorporating an emollient into the vapour permeable viscoelastic polymer gel, the material of the present invention may be used to protect and improve the condition of the skin.

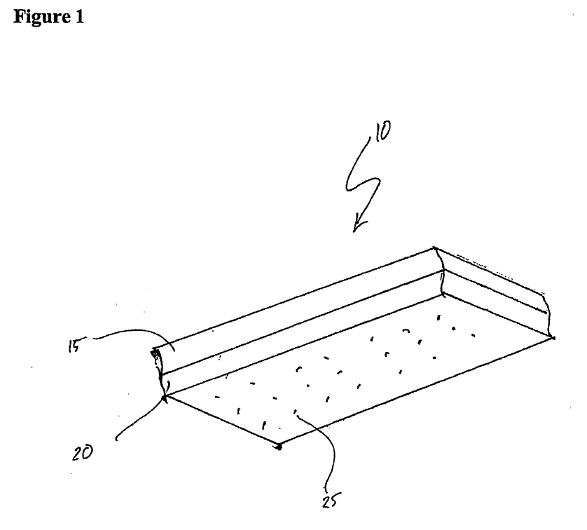

Referring now to FIG. 1 there is shown an example of a section of liquid impermeable composite material 10 including a first layer of material 15 and a second layer of material 20. The second layer of material 20 is composed of a vapour permeable viscoelastic polymer gel, which has been bonded to the first layer of material 10. The first layer of material 10 in this instance is composed of kangaroo leather which is vapour permeable whilst still inhibiting water from passing through the material. The addition of the viscoelastic polymer gel layer does not effect the malleability of the kangaroo leather whilst providing the added characteristic of absorbing the impact of loads and shear forces placed upon the first layer 15.

The second layer 20 includes many perforations 25 that are mechanically provided into the viscoelastic polymer gel. These perforations 25 do not extend to the first layer 15, but provide a passage for vapour passing through the viscoelastic polymer gel providing breathability for the entire composite material 10. Although, the perforations 25 do not allow the passage of liquid such as water to pass through the second layer 20.

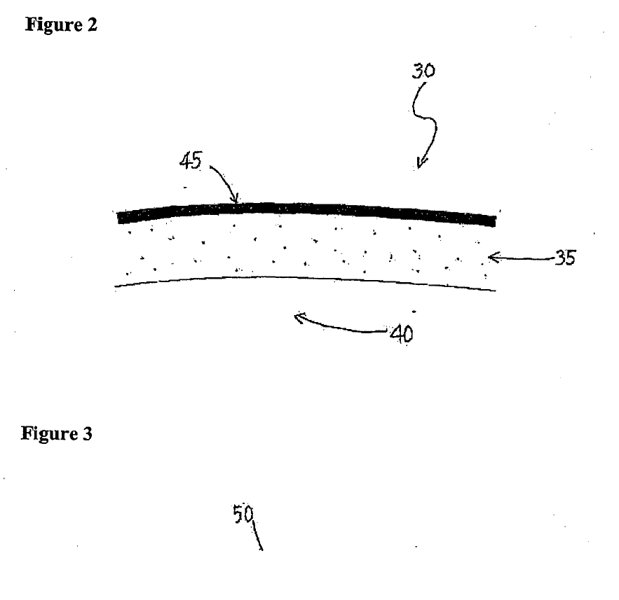

Referring to FIG. 2 there is shown an enlarged cross section of a composition of a ball 30 for playing sports such as soccer or football. The first layer 35 may be composed of a material such as leather, vinyl or the like and the second layer 35 is composed of a vapour permeable viscoelastic polymer gel. Within the first and second layers, a typical inflatable inner may be found 40.

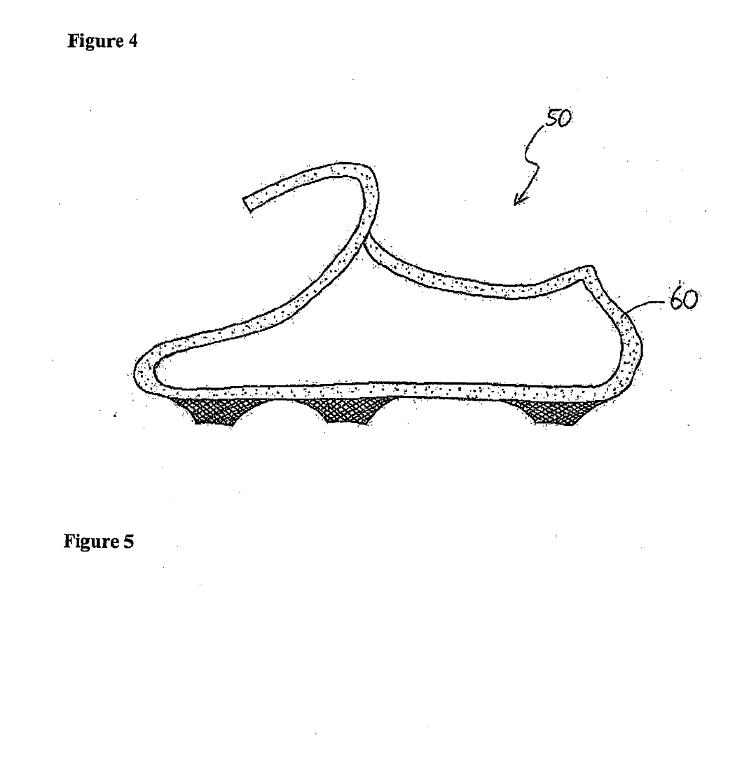

Referring to FIGS. 3 and 4, there is depicted a general outline of an article of footwear 50 which in the particular embodiment is shown in the form of a football boot. As in the case of football boots, the main area of contact 5 in the upper portion of the football boot is reinforced with a liquid impermeable material outlined above. This provides that shear stresses and any impacts are absorbed by the material protecting the foot of the wearer. The liquid impermeable material may be used throughout the construction of the entire article of footwear 50, such that it behaves like a sock around the wearer's foot to protect from shear forces and impacts that may be placed on the article 50, whilst still allowing breathability and providing a water proof environment.

Referring specifically to FIG. 4, the inner sole portion of the boot can be composed entirely of the viscoelastic polymer gel, and may further include a textured surface immediately in contact with a wearer's foot. Such a textured surface provides a better feel for the wearer similar to that when running with bare feet. Such an arrangement allowing this sensation for the wearer has been shown to reduce the likelihood of foot and ankle injuries when active.

FIG. 5 depicts a mechanical apparatus 75 that may be used at the end of the formation of the liquid impermeable material 80 to provide perforations through the viscoelastic gel 85. These perforations are sufficiently small to allow vapour permeability through the viscoelastic gel 85 whilst restricting the movement of any liquid material therethrough. The apparatus 75 is in the form of a roller with many needles 90 protruding from around its circumference. The device is rolled over the viscoelastic gel 85 and the needles pierce the gel and leave perforations without impacting on the first layer of the material 95.

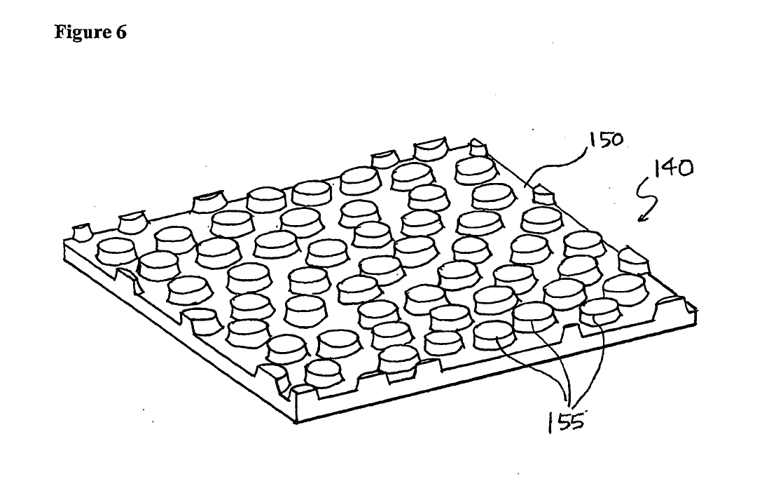

FIG. 6 depicts a piece of material 140 consisting of the viscoelastic polymer gel in accordance with the present invention. The top surface 150 of the viscoelastic includes a textured surface that comprises of circular nodules 155. Such an embodiment of the material of the present invention finds particular utility when used as the contact surface for a persons foot within a shoe construction. The textured surface provides a better touch sensation to the person wearing the shoe such that the experience is similar to walking on a surface with bear feet.

Such nodules may be incorporated into the material of the present invention during formation by extruding the material in its liquid heated state onto a die including the textured pattern. After the material of the present invention cools, you are left with a viscoelastic polymer gel with a textured surface.

The present invention will become better understood from the following examples of preferred but non-limiting embodiments thereof:

### Examples 1 and 2

Polymer component B consisting of polyisoprene was heated for approximately 10 to 15 minutes at 200° C. until completely liquefied. Polymer B was heated first as it takes longer to reach the liquefied state than Polymer A in the circumstances of this example. Polymer component A consisting of up to 80% ethylene propylene styrene block copolymers and mineral or carrier oils ionically polymerised, is added to the liquefied Polymer component B. The resulting mixture is then heated for a further 5 to 10 minutes until liquefied, and a homogeneous melt is achieved. Anhydrous lanolin 5% weight is then added to further reduce viscosities, whilst providing an excellent skin emollient. The 3A molecular sieve having selectivity for water vapour molecules is then added at 20% weight and thoroughly mixed. Finally, 5% hydrocerol CF (nucleating agent) is added and completely mixed at 210 to 220° C. to achieve maximum gas release for a further 2 to 5 minutes (depending on batch size and mixer capability). The mix was then pumped to a die with a heated doctor knife that traverses the casting table or over a roller for coating to the predetermined thickness. In this case, 1 and 2 mm thicknesses for the viscoelastic polymer sheet were chosen. The liquefied melt was then laid undisturbed until it reached room temperature and set, usually within 5 minutes.

Samples of the material of 1 mm & 2 mm were then tested for Mass Vapour Transfer Rates (MVTR) using the test procedure standard: JIS L-1099 B-1(2layer)

### Results: MVTR for Each Sample

| | |
|---|---|
| 1 mm Stretched 50% | 2500 g/m² · 24 hrs |
| 1 mm Unstretched | 500 g/m² · 24 hrs |
| 2 mm Stretched 50% | 1000 g/m² · 24 hrs |
| 2 mm Unstretched | <500 g/m² · 24 hrs |

The results show that the resultant viscoelastic polymer gel was substantially vapour permeable at both 1 and 2 mm thicknesses.

### Examples 3-8

A viscoelastic polymer gel was prepared as in Examples 1 & 2, 1 and 2 mm samples were then taken as above, and each passed through a microperforation process with various quantities of perforations per cm². The MVTR for each sample was then calculated as in the case above in Examples 1 and 2.

### Results

| | | |
|---|---|---|
| 50 pores/cm² 1 mm material | 4000 | g/m² · 24 hrs |
| 100 pores/cm² 1 mm material | 8000 | g/m² · 24 hrs |
| 150 pores/cm² 1 mm material | >12,500 | g/m² · 24 hrs |
| 50 pores/cm² 1 mm material | 1000 | g/m² · 24 hrs |
| 100 pores/cm² 1 mm material | 4000 | g/m² · 24 hrs |
| 150 pores/cm² 1 mm material | 7000 | g/m² · 24 hrs |

The results show that the resultant viscoelastic polymer gel was even further vapour permeable at both 1 and 2 mm thicknesses than that of the material produced in Examples 1 and 2.

### Examples 8-10

In these examples the formulation of examples 1 & 2 are used then before extruding through the die up to 5% deodoriser in powder or oil form is added, then the normal pumping and setting methods applied.

Colorants to 3% that are biologically safe and inert were also added or special effects additives that provide reflections and shine are also added at this stage, mixed evenly a further 5-10 minutes then the material is formed out or extruded.

### Examples 11-12

The viscoelastic polymer is formulated without a nucleating agent and 3A sieve as detailed in Examples 1 and 2. In addition, a flame retardant Aluminium Trihydroxide at 40% by weight is thoroughly blended for 15 minutes, and then extruded into a sheet of any thickness. This produced a flame retarded, low smoke toxicity and low smoke opacity material without significantly altering the properties of the viscoelastic gel material.

Utilising 50% by weight microencapsulated (mPCM) in the 18-28° C. temperature range produces a material that has thermoregulatory properties that increase comfort against human tissue. Lower temperature range mPCM's were also blended to result in a material that distinctly cools human tissue in the range of 4-8° C. Again without altering the mechanical properties of the viscoelastic gel material significantly.

### Example 13

Is a control sample that is produced by Polymer A and B when liquefied and blended together having cooled and formed into a useable sheet, but with none of the various additives or treatments described in reference to examples 1 to 12. Samples were taken at 1 or 2 mm thicknesses and tested but were found to have no significant MVTR, thermoregulation, flame and/or fire retardancy.

Finally, it can be understood that the inventive concept in any of its aspects can be incorporated in many different constructions so that generality of the preceding description is not superseded by the particularity of the attached drawings. Various alterations, modifications and/or additions may be incorporated into the various constructions and arrangements of parts without departing from the spirit or ambit of the present invention.

1. A liquid impermeable material comprising a layer of vapour permeable viscoelastic polymer gel.

2. A material according to claim 1 wherein the vapour permeability is provided by mechanically perforating the viscoelastic polymer gel.

3. A material according to claim 1 wherein the mechanical perforation provides microperforations of 10 to 150 μm in diameter and from 50 to 400 microperforations per cm² of the viscoelastic polymer gel layer.

4. A material according to claim 1 wherein the vapour permeability is provided by incorporating a foaming or nucleating agent into the viscoelastic polymer gel during formation.

5. A material according to claim 4 wherein the amount of foaming or nucleating agent incorporated into the viscloelastic polymer gel during formation is between 0.5 to 20% wt.

6. A material according to claim 4 or claim 5 wherein the foaming or nucleating agent is Hydrocerol CF.

7. A material according to claim 1 wherein the vapour permeability is provided by incorporating a molecular sieve into the viscoelastic polymer gel during formation.

8. A material according to claim 7 wherein the molecular sieve is a 3A molecular sieve.

9. A material according to claim 1 wherein the vapour permeability is provided and/or enhanced by one of the following processes, or a combination thereof:
(a) mechanically perforating the viscoelastic polymer gel layer;
(b) incorporating a foaming or nucleating agent into the viscloelastic gel during formation; and/or,
(c) incorporating a 3A molecular sieve into the viscoelastic polymer gel during formation.

10. A material according to any one of the proceeding claims wherein the composition of the viscoelastic polymer gel includes:
(a) 45% to 100% wt major polymer component A, and
(b) optionally 5% to 45% wt minor polymer component B,
wherein major polymer component A is chosen from any vinyl or polyolefin polymer, or combination thereof, that forms a viscloelastic polymer gel, and component B is chosen from any diene polymer or polyvinyl alcohol.

11. A material according to claim 10 wherein major polymer component A is chosen from any linear or branched polyolefin group, such as for example ethylene, styrene or propylene, or a combination thereof.

12. A material according to claim 11 wherein the major polymer component A consists of up to 80% ethylene propylene styrene block copolymers and mineral or carrier oils ionically polymerised.

13. A material according to claim 10 wherein the minor polymer component B is chosen from polyisoprene.

14. A material according to any one of claims 10 to 13 wherein the ratio of major polymer component A to minor polymer component B is 80:20.

15. A material according to any one of claims 10 to 14 wherein the composition further includes 0.5% to 10% wt lanolin.

16. A material according to any one of the proceeding claims wherein the thickness of the viscoelastic polymer gel layer is from 0.25 mm to 10 mm.

17. A material according to claim 16 wherein the thickness of the viscoelastic polymer gel layer is from 0.5 mm to 3 mm.

18. A material according to any of the preceding claims wherein the viscoelastic polymer gel layer has a Shore Hardness value in the range of 25 to 75.

19. A material according to any of the preceding claims wherein the viscoelastic polymer gel layer has a tensile strength greater than 60 psi.

20. A material according to any of the preceding claims wherein the viscoelastic polymer gel layer has an ultimate elongation of between 250 and 1700%.

21. A composite material which includes:
(a) a first layer; and,
(b) a second layer consisting of a liquid impermeable vapour permeable material according to any one of claims 1 to 20.

22. A composite material according to claim 21 wherein the second layer is bonded to the first layer.

23. A composite material according to claim 21 or 22 wherein the first layer is composed of any natural or man made material that is used in the textile industries, such as for example, plastics, leather, vinyl, cotton, synthetic microfibres, rubber and wool.

24. A composite material according to any one of claims 21 to 23 wherein the first layer is composed of a material that is vapour permeable.

25. A composite material according to any one of claims 21 to 24 wherein the first layer is composed of leather.

26. A composite material according to any one of claims 21 to 25 wherein the first layer is a premium leather such as for example kangaroo leather.

27. An article of footwear including an upper portion composed of a composite material according to any one of claims 21 to 26.

28. An article of footwear according to claim 27 wherein the article is designed as a football boot for use in relation to soccer, rugby union, Australian rules football, rugby league, American football and the like, or any type of football which involves a high level of impact loads and shear stresses being applied to the foot surrounded by the upper portion of the article of footwear.

29. An article of footwear according to claim 27 or claim 28 wherein the composite material is used in relation to any portion of the article that is in contact with the foot of a wearer.

30. An article of footwear according to claim 29 wherein insole of the article in contact with the bottom of the wearer's foot is composed of the material according to any one of claims 1 to 18.

31. An article of footwear according to claim 30 wherein the material includes a textured pattern thereon.

32. A ball for use in playing sport including an outer surface portion composed of a composite material according to any one of claims 21 to 26.

33. A bandage for use in medical, therapeutic and/or household applications composed of the material according to any one of claims 1 to 20.

34. A lining for a bandage and/or a cast composed of the material according to any one of claims 1 to 20.

35. Use of the material according to any one of claims 1 to 20 in medical and/or therapeutic applications requiring a vapour permeable and liquid impermeable material that is hypoallergenic.

36. An article of swimwear composed of the material according to any one of claims 1 to 20.

37. A glove composed of the material according to any one of claims 1 to 20.

38. A composite material including the material of any one of claims 1 to 20 bonded to a film or scrim of hydrophilic nature.

39. A composite material according to claim 38 wherein the film or scrim is composed of polyester, polyethylene and/or polyurethane.

40. A method of producing a liquid impermeable vapour permeable material composed of a viscoelastic polymer gel layer including the following steps:
heating a major polymer component A to the molten state;
optionally adding anhydrous lanolin;
optionally adding a foaming or nucleating agent;
optionally adding a minor polymer component B and mixing the resultant mixture;
optionally adding a 3A molecular sieve;
extruding the resulting mixture into a flat sheet of material of desired thickness and allowing to cool and form a viscoelastic polymer gel; and,
optionally mechanically perforating the sheet.

41. A method according to claim 40 that includes a further heating step after the sheet of material has been allowed to cool, wherein the further heating step provides a second gas yield/nucleating effect within the material to occur.

42. A method according to claim 41 wherein a molecular sieve can be additionally layered/spread or dusted over the material before the further heating step.

43. A method according to any one of claims 40 to 42 wherein the major polymer component A is chosen from any linear or branched polyolefin group, such as for example ethylene, styrene or propylene, or a combination thereof.

44. A method according to any one of claims 40 to 43 wherein the major polymer component A consists of up to 80% ethylene propylene styrene block copolymers and mineral or carrier oils ionically polymerised.

45. A method according to any one of claims 40 to 44 wherein the minor polymer component B is chosen from polyisoprene.

46. A method according to any one of claims 40 to 45 wherein the ratio of major polymer component A to minor polymer component B is 80:20.
